(19) United States
(12) Patent Application Publication
(10) Pub. No.: US 2005/0287216 A1
(43) Pub. Date: Dec. 29, 2005

(54) Medical imaging agents for injectable compositions

(76) Inventor: Gary L. Loomis, Rancho Santa Fe, CA (US)

Correspondence Address: G. L. LOOMIS & ASSOCIATES, INC., 990 HIGHLAND DRIVE, SUITE 212Q, SOLANO BEACH, CA, 92075, US

(21) Appl. No.: 10/880,229

(22) Filed: Jun. 29, 2004

(51) Int. Cl. A61K 9/14

(52) U.S. Cl. 424/486

(57) ABSTRACT

Medically useful compositions, methods and devices comprising insoluble particulate imaging agents are described. The individual particles of the imaging agents of these compositions, methods and devices exhibit regular, smooth morphologies and uniform particle size distributions that allow for enhanced endovascular delivery of the compositions to a mammalian body.

### FIELD OF THE INVENTION

The invention is directed to insoluble particulate imaging agents that impart enhanced performance characteristics to medically useful fluid compositions.

### BACKGROUND OF RELATED ART

In the medical arts the development of microcatheters and guide wires capable of providing access to blood vessels of less than 1 mm diameter allows for the endovascular treatment of a variety of diseases states. In this regard, recent advancements in catheter technology as well as in angiography and medical imaging now permit endovascular intervention for the treatment of otherwise inoperable lesions such as arteriovenous malformations, cerebral aneurysms, fistulas and tumors.

Embolization is a medical procedure resulting in the intentional blockage of a blood vessel to restrict or completely stop the flow of blood through that vessel. Such embolization procedures are accomplished via catheter techniques that permit the selective placement of the catheter at the site to be embolized and for the precise delivery of the embolic device. Embolization procedures may be used to treat a variety of conditions such as organ bleeding, gastrointestinal bleeding, vascular bleeding, bleeding associated with an aneurysm, or to ablate diseased tissue. Also, uterine artery embolization (UAE) has recently emerged as a primary therapy for the treatment of benign fibroid tumors of the uterus and offers a minimally invasive alternative to surgery. The embolic devices employed in these procedures include liquid or fluid systems of the types described in U.S. Pat. No. 5,851,508; U.S. Pat. No. 6,476,069 and U.S. Pat. No. 6,476,070.

Devices and compositions for use in endovascular treatment regimens ideally include imaging agents so that the practitioner, aided by a suitable imaging technique, can visualize the delivery of the composition or device to the vascular site. Such imaging agents are also known in the art as contrast agents. When the imaging agent is radiopaque, x-ray techniques such as fluoroscopy may be used for visualization. Visualization is particularly necessary when using catheter delivery techniques in order to ensure both that the composition or device is being delivered to the intended vascular site and that the requisite quantity of material is delivered. Many radiopaque imaging agents are insoluble in blood or other body fluids and the use of such insoluble imaging agents is beneficial during post-treatment procedures in order to visualize the embolized mass during, for example, surgery or to monitor the disease condition for re-treatment purposes.

Often the insoluble imaging agents used in these applications are particles of non-reactive metal such as tantalum (TRUFILL° n-BCA Liquid Embolic System from Cordis Neurovascular, Inc., Miami Lakes, Fla.). Also, U.S. Pat. No. 5,851,508 describes medically useful liquid compositions containing a metal or metal oxide as a water-insoluble radiopaque imaging agent. The use of high-purity gold as a radiopaque imaging agents in a liquid embolic systems has been described in U.S. Pat. No. 6,476,069 and U.S. Pat. No. 6,476,070. The use of metallic powders such as barium or tantalum to render liquid embolic compositions radiopaque is described in U.S. Pat. No. 6,296,604. Insoluble metal-cation salts of anionic polymer are described in U.S. Pat. No. 5,702,682 for use as insoluble radiopaque imaging agents in medical devices.

Systems containing insoluble paramagnetic radiopaque particles in flowable embolic systems are described in U.S. Pat. No. 6,364,823. Such compositions render the embolic material magnetic allowing it to be controlled, directed, deposited, and held in place with an external magnetic field.

The particulate materials commonly used as insoluble radiopaque imaging agents in the liquid or fluid systems as described above are generally produced by processes involving grinding, milling, or other manner of mechanical particle-size reduction. The particles resulting from these processes are generally irregular in shape and exhibit a broad particle size distribution. As a result of the irregular morphology of these materials it is known that the particles can aggregate and cause clogged and damaged catheters during injection. Premature aggregation of the particles may also result in blockage of vital blood flow to healthy tissue. Furthermore, the tendency of these particulate materials to aggregate imposes a practical limit on the concentration of the insoluble imaging agent that can be used in a given composition. Compositions containing high-levels of particulate imaging agents are often difficult or impossible to impel through the delivery catheter and it has been observed that the forcing of such a composition through a micro-catheter can cause rupture of the catheter wall with disastrous consequences. Also, as a consequence of broad particle size distribution, it is often difficult to fully disperse particulate imaging agents in the liquid compositions.

Thus, a need exists in these applications for improved insoluble particulate imaging agents that prevent aggregation of the particles within the catheter lumen or in the vasculature and promote ease and accuracy of delivery. Also, there exists a need for liquid embolic compositions that contain higher concentrations of insoluble imaging agents than the concentrations used in compositions currently described and used in the art. Finally, there exists a need for insoluble imaging agents that are more easily dispersible in medically useful fluid compositions. The present invention is directed to meeting these and other needs.

### SUMMARY OF THE INVENTION

This invention is directed to syringe-deliverable or catheter-deliverable compositions containing particulate imaging agents such compositions being useful in therapeutic treatments involving endovascular access to a mammalian body. More specifically, invention provides medically useful fluid compositions containing insoluble particulate imaging agents, wherein such the individual particles of such imaging agents exhibit a regular, smooth morphology and a uniform particle size distribution so as to effect the enhanced performance of these compositions.

Embodiments the compositions, methods and medical devices of the present invention are useful for filling or partially filling various intravascularly accessible body cavities in mammalian bodies. In one embodiment the compositions are useful for the embolization of blood vessels. In another embodiment the compositions are useful for treating vascular aneurysms where only the aneurismal sac filled with the composition while leaving the adjoining blood vessel unaffected. In still another embodiment the compositions are useful for treating endoleaks arising from endovascular repair of abdominal aortic aneurysms with stent grafts.

### DETAILED DESCRIPTION

The present invention is directed to medically useful compositions comprising insoluble particulate imaging agents in which the individual particles exhibit a regular, smooth morphology and uniform particle size distributions. Such imaging agents allow for enhanced intravascular delivery of these compositions. More specifically, this invention is directed to syringe-deliverable or catheter-deliverable compositions useful in therapeutic treatments involving endovascular access to a mammalian body.

The terms imaging agent and contrast agent both refer to a material capable of being monitored by a suitable imaging method during a procedure involving injection of the material into a mammalian body. For the purposes of the present invention the terms imaging agent and contrast agent are synonymous. If the imaging agent is a radiopaque material, the procedure can be monitored by an x-ray imaging techniques such as fluoroscopy.

If the imaging agent is a paramagnetic material the procedure can be monitored by imaging techniques such as nuclear magnetic resonance imaging (MRI). A paramagnetic material is a material that is attracted by a magnetic field, but does not retain magnetism once the magnetic field is removed. Additionally, the presence of the paramagnetic particles allows a fluid composition to be directed, deposited, and held in place with a magnetic field. In some medical procedures the paramagnetic particulate material is a magnetic powder such as pure iron, carbonyl iron, coated iron and coated carbonyl iron (preferably pure iron) and it is used for both radiopacity and magnetic attraction. In one embodiment, the material becomes less magnetically responsive over time so that the presence of the material does not interfere with or restrict subsequent magnetic procedures such as magnetic surgical procedures or MRI.

In the compositions, methods and devices of the present invention all materials described as insoluble exhibit a solubility in either water, saline, blood or other body fluid of less than 0.01 mg/ml at 20° C. as well as a solubility of less than 0.01 mg/ml at 20° C. in the liquid component(s) of the compositions, methods or devices.

A list of insoluble particulate imaging agents useful in embodiments of the present invention includes, but is not limited to, inorganic compounds such as tantalum oxide, bismuth trioxide, barium sulfate and the like; metal powders such as tantalum, gold, tungsten, platinum, palladium, and silver as well as mixtures and alloys thereof; brominated or iodinated organic compounds; and brominated or iodinated organic polymers. In other applications the particles may be paramagnetic metal salts or chelates. In certain cases it may be desirable that the particulate material be radioactive. Embodiments of the present invention are in no way limited to the materials described here and additional materials useful as imaging agents for the practice of the invention will be apparent to those skilled in the art.

The particle size reduction of commonly produced particulate materials, such as metals, metal salts, metal oxides, ceramics and polymers is normally achieved by processes involving grinding or milling. Therefore, the particles resulting from such particle size reduction processes tend to be irregular in shape and exhibit a broad particle size distribution.

By design, the particulate materials useful in embodiments of the present invention exhibit a regular morphology with essentially rounded edges and a smooth surface. In preferred embodiments, the insoluble particulate component exhibits a spherical or ellipsoidal morphology with a spherical or nearly spherical morphology being most preferred.

In general, embodiments of the compositions, methods or devices of the present invention utilize insoluble particulate components in which the individual particles have a mean particle diameter of 100 microns or less. In preferred embodiments, the individual particles have a mean particle diameter of 10.0 microns or less and in most preferred embodiments the individual particles have a mean particle diameter of 1.0 micron or less.

Useful in the present invention are spherical particles of noble metals such as gold, platinum, palladium and the like. Such particles may be prepared by chemical processes involving the controlled reductive precipitation of the metal from a solution of a suitable metal salt. A process for the preparation of spherical noble metal particles is described in U.S. Pat. No. 3,930,845. High-purity metal powders exhibiting spherical or ellipsoidal morphology may also be prepared by processes involving atomization as described in U.S. Pat. No. 4,479,823.

Particularly useful in embodiments of the present invention are the precipitated high-purity spherical noble metal powders available commercially from Technic Inc., Engineered Powders Division, Woonsocket, R.I.

The terms fluid composition, liquid composition, and flowable composition, as used in the present invention, refer to either pure liquids, solutions, emulsions, or solid-in-liquid suspensions that can be injected with a syringe or delivered through medical catheters.

In general, the present invention provides methods and compositions useful for filling or partially filling a volume or space in a mammalian body. In particular, the methods and compositions are useful for filling an existing space or volume such as the lumen of a blood vessel or the sac of an aneurysm. Also the methods and compositions are useful for filling a space or volume created by a transiently placed external device such as a catheter or like device or a space created by a medical procedure such as an excision or like procedure or implantation of an object, e.g., a stent or like device. Certain of the compositions of the present invention are transformed into solids when delivered to the desired mammalian body space or volume

Embolization is a medical procedure resulting in the intentional occlusion or partial occlusion blood vessel. Such embolization procedures may be used to treat a variety of conditions such as organ bleeding, gastrointestinal bleeding, vascular bleeding, bleeding associated with an aneurysm, or to ablate diseased tissue such as tumors, uterine fibroids and the like. Transcatheter arterial embolization (TAE) has been shown to be very effective as a treatment for renal cell carcinoma as a non-surgical option. Furthermore, renal cell carcinoma has also been treated successfully with embolic agents prior to surgical excision of the kidney and palliative embolization of inoperable renal tumors with serious hemorrhage has also been successful. Embolization is are often accomplished by use of fluid or liquid embolic compositions that solidify upon introduction into the desired site in a mammalian body. The controlled solidification of such fluid embolic compositions at the desired site in a mammalian body is essentially equivalent to the implantation of a medical device.

In addition to the aforementioned embolization procedures, fluid or liquid embolic compositions can be used for the treatment of endoleaks arising from endovascular repair of abdominal aortic aneurysms with stent-graft devices. These methods provide for delivery of fluid compositions to the site of an endoleaks in the abdominal aorta wherein the fluid composition forms a coherent solid mass that adheres to the vascular wall and wall of the prosthesis with the effect of sealing the endoleak.

Liquid embolic systems fall into two general types. The first type of liquid embolic system, which is herein designated as a Type I liquid composition, is described in U.S. Pat. No. 6,476,069 and U.S. Pat. No. 6,476,070. Such a type I system employs polymerizable monomers, oligomers or pre-polymers including alkyl cyanoacrylates. In these Type I systems the liquid monomers polymerize upon contact with anionic body fluids such as blood resulting in the in situ formation of a solid polymeric medical device that embolizes the blood vessel. An insoluble particulate imaging agent, which is initially suspended in the liquid composition as it is delivered to the body, is ultimately incorporated into the solid medical device.

The second type of liquid embolic composition herein designated as a Type II liquid composition and is described in U.S. Pat. No. 5,851,508 and U.S. Pat. No. 6,017,977. These Type II systems contain a biocompatible solvent, a biocompatible polymer and an insoluble particulate imaging agent. The biocompatible polymer in these Type II systems is selected to be soluble in the biocompatible solvent but insoluble in blood or other body fluid. The biocompatible solvent in these type II systems is miscible or soluble in blood or other body fluid and also solubilizes the biocompatible polymer during delivery. The insoluble imaging agent is suspended in the composition and, as above, permits the practitioner to visualize catheter delivery of the composition. Upon contact with the blood or other body fluid, the biocompatible solvent dissipates from the embolic composition whereupon the biocompatible polymer precipitates in the presence of the insoluble imaging agent and forms a solid medical device that embolizes the blood vessel. As in the Type I system, the insoluble particulate imaging agent of these Type II systems is initially suspended in the liquid composition as it is delivered to the body, and is ultimately incorporated into the solid polymeric medical device.

The medical devices resulting from the solidification of compositions of either type I or type II may be formulated by one skilled in the art to be biostable, partially bioresorbable or totally bioresorbable. Particularly useful as bioresorbable imaging agents are iodinated polyesters, polyester urethanes and other such polymers.

Embodiments of type I liquid compositions comprise one or more polymerizable monomers, oligomers or pre-polymers. Such polymerizable monomers, oligomers or pre-polymers may be anionically polymerizable, free-radical polymerizable, or polymerizable by zwitterions or ion pairs. Such monomers are disclosed in U.S. Pat. No. 5,328,687 which is hereby incorporated in its entirety by reference herein. In such systems the liquid monomers oligomers or pre-polymers polymerize upon contact with body fluids such as blood resulting in the in situ formation of a solid polymer.

Useful polymerizable monomers in the embodiment of type I liquid compositions are 1,1-disubstituted ethylene monomers of the formula (I)
RHC═CXY (I)
wherein X and Y are each strong electron withdrawing groups, and R is H, —CH.═CH₂; or, provided that X and Y are each cyano groups, a C, to C₄ alkyl group.

Examples of monomers within the scope of formula (I) include 2-cyanoacrylates, vinylidene cyanides, C₁-C₄ alkyl homologues of vinylidene cyanides, dialkyl methylene malonates, acylacrylonitriles, vinyl sulfinates and vinyl sulfonates of the formula II
H₂C═CX′Y′ (II)
wherein X′ is —SO₂R′ or —SO₃R′ and Y′ is —CN, —COOR′, —COCH₃, —SO₂R′ or —SO₃R′, and R′ is H or hydrocarbyl.

Monomers of formula (I) useful in embodiments of the present invention are the 2-cyanoacrylate monomers which are known in the art and have the formula (III) 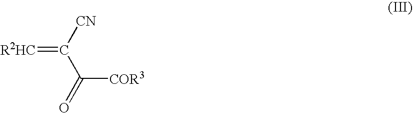
wherein R² is hydrogen and R³ is a hydrocarbyl or substituted hydrocarbyl moiety; a group having the formula —R⁴—O—R⁵—O—R⁶, wherein R⁴ is a 1,2-alkylene group having 2 to 4 carbon atoms, R⁵ is an alkylene group having 2 to 4 carbon atoms, and R⁶ is an alkyl group having 1 to 6 carbon atoms; or a group having the structure
—R⁷—CH₂—O—R⁸
wherein R⁷ is 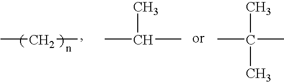
wherein n is 1 to 10, preferably 1 to 5 carbon atoms and R⁸ is an organic moiety.

Also useful in embodiments of Type I liquid compositions are 2-cyanoacrylates monomers of formula (III) wherein R³ is a poly(alkylene) oxide. Such poly(alkylene) oxides can include, for example, poly(ethylene) oxide, poly(propylene) oxide, poly(butylene oxide), and mixtures and copolymers thereof.

The 2-cyanoacrylates of formula (III) can be prepared according to methods known in the art. U.S. Pat. No. 2,721,858 and U.S. Pat. No. 3,254,111, each of which is hereby incorporated in its entirety by reference, disclose methods for preparing 2-cyanoacrylates. For example, the 2-cyanoacrylates can be prepared by reacting an alkyl cyanoacetate with formaldehyde in a non-aqueous organic solvent and in the presence of a basic catalyst, followed by pyrolysis of the anhydrous intermediate polymer in the presence of a polymerization inhibitor. The 2-cyanoacrylates monomers prepared with low moisture content and essentially free of impurities are preferred for biomedical use.

Examples of 2-cyanoacrylate monomers useful in the embodiments of the present invention relating to type I liquid embolic compositions are alkyl 2-cyanoacrylates including, but not limited to, ethyl 2-cyanoacrylate; n-butyl cyanoacrylate; iso-butyl 2-cyanoacrylate; n-hexyl 2-cyanoacrylate; 2-hexyl 2-cyanoacrylate; n-octyl 2-cyanoacrylate; 2-octyl 2-cyanoacrylate; 2-ethylhexyl 2-cyanoacrylate; 3-methoxybutyl 2-cyanoacrylate; 2-butoxyethyl cyanoacrylate; 2-isopropoxyethyl 2-cyanoacrylate; and 1-methoxy-2-propyl 2-cyanoacrylate.

Other monomers useful in the embodiments of the present invention relating to type I liquid embolic compositions are 3-(acryloyloxy)sulfolanes and 3-(methacryloyloxy)sulfolanes of the formula (IV) 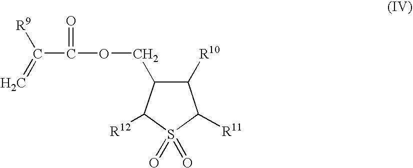
wherein R⁹ is H or CH₃; and wherein R¹⁰, R¹¹, R¹² are either H or organic moieties.

In yet another embodiments of the present invention relating to type I liquid embolic compositions are 3-(acryloyloxy)sulfonates monomers of the formula V 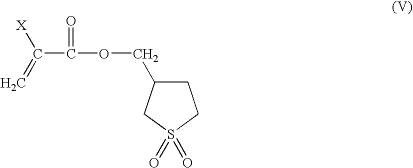
wherein X is —CN, —Cl, —Br, —I, —COCH₃, —COOR′ and R′ is H or hydrocarbyl.

In certain embodiments of the present invention the type I liquid compositions further comprise one or more plastcizers. The term plasticizer in the context of the present invention is to be construed as any material which is soluble or dispersible in a polymerizable composition, and which increases the flexibility of the polymer obtained from polymerization of said polymerizable composition. Such plasticizers must be biocompatible to the extent required for the intended medical application. For example, a plasticizer used in a coating on the skin surface should be compatible with the skin as measured by the lack of skin irritation and a plasticizer used for an implant in the body should be non-toxic or of a toxicity sufficiently low as to be tolerated by the body. Suitable plasticizers are well known in the art and include those disclosed in U.S. Pat. Nos. 2,784,127 and 4,444,933 the disclosures of both of which are incorporated herein by reference in their entirety.

A list of plasticizers useful in compositions, processes and devices of the present invention includes, but is not limited to, fatty acid esters, citrate esters, phthalate esters, benzoate esters, and certain aromatic phosphate esters. By way of example, such useful plasticizers include butyl benzyl phthalate, dibutyl phthalate, diethyl phthalate, dimethyl phthalate, dioctyl phthalate, 2-ethylhexyl phthalate, benzoate esters of di- and poly-hydroxy branched aliphatic compounds, tri(p-cresyl) phosphate, alkyl myristates and the like. Plasticizers particularly useful in this invention are acetyltriethyl citrate, acetyl tri-n-butylcitrate, acetyltri-n-hexyl citrate, and n-butyryltri-n-hexyl citrate.

Embodiments of the type II liquid composition, comprise biocompatible solvents, a biocompatible polymers and an insoluble imaging agents. In these embodiments the biocompatible polymer is selected to be soluble in the biocompatible solvent but insoluble in blood or other body fluid. The biocompatible solvent is miscible or soluble in blood or other body fluid and also solubilizes the biocompatible polymer during delivery. The insoluble imaging agent is suspended in the composition and, as above, permits the physician to fluoroscopically visualize catheter delivery of this composition. Upon contact with the blood or other body fluid, the biocompatible solvent dissipates from the embolic composition whereupon the biocompatible polymer precipitates in the presence of the insoluble imaging agent and embolizes the blood vessel.

The term biocompatible polymer refers to polymers which, in the amounts employed, are non-toxic, chemically inert, and substantially non-immunogenic when used internally in the patient and which are substantially insoluble in blood. Suitable biocompatible polymers include, by way of example, cellulose acetates (including cellulose diacetate), ethylene vinyl alcohol copolymers, hydrogels (e.g., acrylics), poly(acrylonitrile) and the like. Preferably, the biocompatible polymer is also non-inflammatory when employed in situ.

Preferred biocompatible polymers include cellulose diacetate and ethylene vinyl alcohol copolymer. Cellulose diacetate polymers are either commercially available or can be prepared by art recognized procedures.

Ethylene vinyl alcohol copolymers comprise residues of both ethylene and vinyl alcohol monomers. Small amounts (e.g., less than 5 mole percent) of additional monomers can be included in the polymer structure or grafted thereon provided such additional monomers do not alter the embolizing properties of the composition. Such additional monomers include, by way of example only, maleic anhydride, styrene, propylene, acrylic acid, vinyl acetate and the like.

The ratio of ethylene to vinyl alcohol in the copolymer affects the overall hydrophilicity of the composition, which in turn, affects the relative water solubility of the composition as well as the rate of precipitation of the copolymer body fluids such as blood.

The term biocompatible solvent refers to an organic material liquid at least at body temperature of the mammal in which the biocompatible polymer is soluble and, in the amounts used, is substantially non-toxic. Suitable biocompatible solvents include, by way of example, dimethylsulfoxide, analogues and homologues of dimethylsulfoxide, ethanol, acetone, N-methyl-2-pyrollidinone, ethyl lactate and the like.

Both the type I and type II liquid embolic compositions described above can also be used for the occlusion of aneurysms or peripheral blood vessels wherein the aneurysms or peripheral blood vessels are isolated from the general circulation until occlusion is effected. These procedures utilize one or more balloon catheters to isolate the aneurysms or peripheral blood vessels from the general circulation. Such procedures are described in U.S. Pat. No. 6,096,021.

In one embodiment of the present invention the compositions are used for the embolization of blood vessels resulting in the stoppage of blood flow through the vessels. Such embolization procedures are useful in the treatment of a variety of conditions such as organ bleeding, gastrointestinal bleeding, vascular bleeding, bleeding associated with an aneurysm, or the ablation of diseased tissue such as tumors, uterine fibroids and the like.

In other embodiments the compositions of this invention are used for filling or partially filling various intravascularly accessible body cavities in mammalian bodies. In another embodiment the compositions of this invention are useful in for treating vascular aneurysms such as cerebral aneurysms wherein only the aneurismal sac filled with the composition while leaving the adjoining blood vessel unaffected. In such embodiments the compositions may be used as the sole treatment or the compositions may be used as an adjunct with other aneurysm-treating therapeutic devices such as detachable balloons, detachable coils, stents and the like.

In another embodiment of the present invention the compositions are used for treating the various types of endoleaks arising from endovascular repair of abdominal aortic aneurysms with stent grafts.

Another embodiment of the present invention is a method for sterilizing a female mammal comprising the step of administering an embolic composition to the fallopian tubes thereby preventing the passage of the eggs from the ovaries to the uterus of said female mammal.

In still another embodiment of the present invention the compositions are used for the site-specific endovascular delivery and controlled release of pharmacologically active agents.

Whether an embolizing agent and imaging agent will be suitable in combination to embolize a blood vessel is empirical and substitution of one embolizing agent for another or one imaging agent with may afford compositions having different chemical and/or physical properties. However, a common property of the compositions of the present invention is that they are easily manually injectable even when the composition contains a high concentrations of insoluble imaging agent.

The force required to manually depress the plunger of a syringe so as to inject a fluid composition through a catheter depends upon many factors including the viscosity of the liquid composition, the lubricity of the liquid to the syringe and catheter materials, and the capacity and geometries of the delivery syringes and catheters. The precise viscosity and lubricity requirements of the injectable compositions in a given situation will be apparent to those skilled in the art and the compositions may be modified and formulated as necessary. Injectable compositions with imaging agent concentrations as high as 5.0 g of gold to 1.0 ml of a type I liquid embolic have been observed to be manually injectable with a 3.0 ml syringe equipped with a 2 inch-20 gauge needle.

The invention is further describe with reference to the following examples which are provided for purposes of illustration and are not intended to be limiting in any sense.

### EXAMPLES

Gold powder was obtained from Technic, Inc., Engineered Powders Division, Woonsocket, R.I. Tantalum was obtained as component of a TRUFILL® n-BCA Liquid Embolic System (Cordis Neurovascular, Inc., Miami Lakes, Fla.). Ethyl myristate (97%, Cat. No. E3 960-0) was obtained from Aldrich Chemical Company.

A syringe pump (KD Scientific Model No. 200) was modified to incorporate a linear force transducer (Cooper Instruments Model No. LPM-530-50) in a manner such that the force required to push the plunger could be measured in real-time during the dispensing of the suspension from the syringe (Display: Cooper Instruments Model No. DFI Infinity CS). Syringes used were Norm-Ject 3 ml plastic syringes with non-elastomeric plunger tips, and were determined to be dimensionally stable when in contact with the ethyl myristate (no swelling). The syringe pump was operated at two settings to deliver ethyl myristate/gold mixtures through the delivery catheters at rates of 10 ml/min and 20 ml/min. Prior to the experiment, a blank was prepared by charging a syringe with ethyl myristate, followed by recording the plunger force encountered during dispensing of the liquid through the catheter.

Polyethylene delivery catheters of 150 cm overall shaft length with an internal diameter of 0.019 in, and dead space of approximately 0.274 ml. were custom manufactured by Modified Polymer Components, Inc., Sunnyvale, Calif. The catheters were rinsed with 10 ml ethyl myristate before introduction of the ethyl myristate/gold mixture and were kept filled until ready for use.

### Example 1

This experiment demonstrates the effects of several types of particulate gold on syringe plunger force. To 5-ml glass serum vials was added 1.0 g of particulate gold and 2.0 ml of ethyl myristate. Each vial was sealed until ready for use and was subsequently agitated with a Thermolyne Mixer (Model No. M16715) for 1.0 min to suspend the gold in the ethyl myristate. The syringe was charged with the gold-in-ethyl myristate suspension and was then connected to the catheter and affixed to the syringe pump. The syringe pump operated at until the gold-in-ethyl myristate suspension was discharged from the tip of the catheter. Peak force was recorded at 30 sec intervals.

Data was obtained with suspensions of spherical gold in ethyl myristate at two dispensing rates. The spherical gold particles used to prepare these suspensions differ only in mean particle diameter. The maximum force (lb/in²) measured for each sample with respect to mean particle diameter (microns) is presented in Table 1.

TABLE 1

| | | | | Plunger |
|---|---|---|---|---|
| Gold Type | Particle | Average | Pump Rate | Force |
| (Technic No.) | Morphology | Diameter (u) | (ml/hr) | (lbs) |
| 12-509 | Spherical | 3.5 | 10 | 2.6 |
| 12-508 | Spherical | 1.8 | 10 | 2.3 |
| 12-505 | Spherical | 1.0 | 10 | 2.1 |
| 12-504 | Spherical | 0.5 | 10 | 1.8 |
| 12-509 | Spherical | 3.5 | 20 | 1.8 |
| 12-508 | Spherical | 1.8 | 20 | 1.9 |
| 12-505 | Spherical | 1.0 | 20 | 1.7 |
| 12-504 | Spherical | 0.5 | 20 | 1.3 |

The data in Table 1 illustrates the relationship between the plunger force required for catheter delivery to the average diameter of the spherical gold particles. The smaller the average particle diameter of the spherical gold particles the lower is the peak plunger force required for catheter delivery of gold-in-ethyl myristate suspensions. This is true at both the high dispensing rate (20 ml/hr) and the low dispensing rate (10 ml/hr). The lowering of the plunger force is advantageous when performing interventional procedures due to increased dispensing control of liquid interventional products such as the liquid embolic compositions of the present invention. Also, increased control of dispensing reduces chances of unwanted or premature material discharging from a delivery catheter during an interventional procedure.

### Example 2

To individual 5-ml glass serum vials was added 1.0 g of particulate metal and 1.0 ml of ethyl myristate. Each vial was sealed until ready for use and was then agitated with a Thermolyne Mixer (Model No. M16715) for 1.0 min to suspend the metal in the ethyl myristate. The syringe was charged with the metal-in-ethyl myristate suspension and then was connected to the catheter and affixed to the syringe pump. The syringe pump was operated until the metal-in-ethyl myristate suspension was observed discharging from the catheter tip. The maximum force (lb/in² and back pressure (lbs/in²) was measured for each sample and the results were evaluated with respect to mean particle diameter and the results are presented in Table 2.

TABLE 2

| | | | Plunger | Back |
|---|---|---|---|---|
| | | Average | Force | pressure |
| Metal Type | Morphology | Diameter (μ) | (lbs) | (lbs/in²) |
| no metal | NA | NA | 0.5 | 2.5 |
| (control) | | | | |
| gold | Spherical | 0.5 | 1.9 | 9.4 |
| gold | Spherical | 1.0 | 1.8 | 8.9 |
| gold | Spherical | 3.5 | 0.5 | 2.5 |
| tantalum | irregular (jagged) | 50.0 | 15.0 | 74.4 |

The data in Table 2 dramatically demonstrate that the dispensing of a suspension comprising a particulate metal of small particle size and spherical morphology requires a lower plunger force than does the dispensing of a suspension comprising a particulate metal of large particle size and irregular, jagged morphology. It is also notable that the back pressure in the syringe is significantly lower for the suspensions comprising the particulate metal of small particle size and spherical morphology.

### Example 3

This example demonstrates the effect of particle morphology on the deliverability of fluid compositions comprising insoluble particulate imaging agents. To 5-ml glass serum vials was added 1.0 ml of ethyl myristate and the requisite amount of metal powder as shown in table 3. Each vial was sealed until ready for use and then agitated with a Thermolyne Mixer (Model No. M16715) for 1.0 min in order to suspend the metal powder in the ethyl myristate. A 3.0 ml syringe was charged with each composition and the composition was immediately dispensed manually through a 12 gauge hypodermic needle using. The practical manual deliverability of the compositions was assessed by an experienced interventional radiologist and is designated as a simple yes or no. The results of the evaluation are presented in Table 3.

TABLE 3

| | | A | B | C | D |
|---|---|---|---|---|---|
| | | Tantalum Jagged | Gold | Gold | Gold |
| | | and Irregular | Spherical | Spherical | Spherical |
| | | (Cook Medical) | (Technic 505) | (Technic 508) | (Technic 509) |
| | | avg. particle diameter (μ) | | | |
| | metal:ethyl myristate | 0.5-1.0 | 0.5-1.0 | 2.0-4.0 | 1.0-2.0 |
| | wt:vol | DELIVERABILITY | | | |
| 1 | 1:1 | yes | yes | yes | yes |
| 2 | 1.5:1 | no | yes | yes | yes |
| 3 | 2:1 | no | yes | yes | yes |
| 4 | 3:1 | — | yes | — | — |
| 5 | 4:1 | — | yes | — | — |
| 6 | 5:1 | — | yes | — | — |
| 7 | 6:1 | — | no | — | — |

The particulate (0.5-1.0 micron) tantalum of column A was observed by scanning electron microscopy (SEM) to have a jagged irregular morphology. By comparison, the particulate (0.5-1.0 micron) gold of column B, which has a similar particle size distribution, was determined by scanning electron microscopy (SEM) to have smooth-surface and essentially spherical morphology. It is apparent from results presented in Table 3 that the compositions of column B were deliverable in metal to ethyl myristate wt:vol ratios as high as 6:1, while the composition s of column A were not deliverable in metal to ethyl myristate ratios higher than 1:1. Furthermore, it is also remarkable that the compositions 1 to 3 of columns C and D, which utilize spherical gold in average particle diameters significantly greater than those of the compositions of column A and column B, were also deliverable at ratios significantly higher than the 1:1 ratio of the composition A1.

1. A medically useful fluid composition comprising an insoluble particulate imaging agent wherein the individual particles of said particulate imaging agent have a smooth and regular morphology.

2. The composition of claim 1 wherein the individual particles of said particulate imaging agent are essentially spherical.

3. The composition of claim 1 wherein the individual particles of said particulate imaging agent have a mean particle diameter less than or equal to 100 microns.

4. The composition of claim 1 wherein the individual particles of said particulate imaging agent have a mean particle diameter less than or equal to 10 microns.

5. The composition of claim 1 wherein the individual particles of said particulate imaging agent have a mean particle diameter less than or equal to 1.0 micron.

6. The composition of claim 1 wherein said insoluble particulate imaging agent component is a metal.

7. The composition of claim 6 wherein said metal is selected from the group consisting of gold, platinum, palladium, tantalum, tungsten, and alloys thereof.

8. The composition of claim 7 wherein said metal is gold.

9. The composition of claim 1 wherein said fluid composition further comprises a polymerizable monomer.

10. The composition of claim 9 wherein said polymerizable monomer is an alkyl 2-cyanoacrylate.

11. The composition of claim 10 wherein said alkyl 2-cyanoacrylate is selected from the group consisting of ethyl 2-cyanoacrylate; n-butyl 2-cyanoacrylate; isobutyl 2-cyanoacrylate; n-hexyl 2-cyanoacrylate; 2-hexyl 2-cyanoacrylate; n-octyl 2-cyanoacrylate; 2-octyl 2-cyanoacrylate; 2-ethylhexyl 2-cyanoacrylate; 3-methoxybutyl 2-cyanoacrylate; and 2-butoxyethyl cyanoacrylate.

12. The composition of claim 11 wherein said alkyl 2-cyanoacrylate is selected from the group consisting of n-hexyl cyanoacrylate; 2-hexyl 2-cyanoacrylate; n-octyl 2-cyanoacrylate; 2-octyl 2-cyanoacrylate; and 2-ethylhexyl 2-cyanoacrylate.

13. The composition of claim 9 wherein said fluid composition further comprises one or more additional polymerizable monomers.

14. The composition of claim 1 wherein said fluid composition further comprises a plasticizer.

15. The composition of claim 1 wherein said fluid composition further comprises a biocompatible polymer and a biocompatible solvent.

16. The composition of claim 15 wherein said biocompatible polymer is selected from the group consisting of cellulose diacetate, cellulose triacetate and ethylene vinyl alcohol copolymer; and said biocompatible solvent is selected from the group consisting of ethanol, acetone, dimethyl sulfoxide and N-methyl-2-pyrollidinone.

17. The composition of claim 15 wherein said biocompatible polymer is ethylene vinyl alcohol copolymer and said biocompatible solvent is dimethyl sulfoxide.

18. The composition of claim 1 further comprising a pharmacologically active agent.

19. A method of filling or partially filling a cavity in a mammalian body comprising the step of introducing intravascularly the composition of claim 1 to said cavity.

20. The method of claim 19 wherein said cavity is a blood vessel.

21. The method of claim 19 wherein said cavity is a vascular aneurysm.

22. The medical device resulting from the solidification of the fluid composition of claim 1 in a cavity of a mammalian body.

23. The medical device of claim 22 wherein said cavity of a mammalian body is a blood vessel.

24. The medical device of claim 22 wherein said cavity of a mammalian body is a vascular aneurysm.
